(19) United States
(12) Patent Application Publication
(10) Pub. No.: US 2019/0350715 A1
(43) Pub. Date: Nov. 21, 2019

(54) LAMINATED STRUCTURE WITH ADJUSTABLE ELASTIC MODULUS

(71) Applicant: Wizdom Inc., Las Vegas, NV (US)

(72) Inventors: Yung-Lung Liu, Kaohsiung City (TW); Ralph Lee, Kaohsiung City (TW); Wen-Yi Chen, Kaohsiung City (TW); Chin-Sheng Chen, Kaohsiung City (TW); Royal Su, Kaohsiung City (TW); Zi-Xun Chen, Kaohsiung City (TW)

(21) Appl. No.: 16/153,179

(22) Filed: Oct. 5, 2018

(30) Foreign Application Priority Data
May 15, 2018 (TW) 107116483

(51) Int. Cl. A61F 2/30; B33Y 80/00

(52) U.S. Cl. CPC A61F 2/30767; B33Y 80/00; A61F 2002/30985; A61F 2002/30971; A61F 2002/3093

(57) ABSTRACT

A laminated structure with an adjustable elastic modulus has multiple connecting elements and at least one connecting point. Each connecting element is an S-shaped curved component, and has an inner end and an outer end. The multiple connecting elements are combined with each other by at least one connecting point, and a diameter of the at least one connecting point is equal to or greater than a diameter of each connecting element connected by the at least one connecting point.

### BACKGROUND OF THE INVENTION

### 1. Field of the Invention

The present invention relates to a medical artificial implant, especially to a laminated structure with an adjustable elastic modulus which is applied to a medical artificial implant.

### 2. Description of Related Art

With the advance of technology, 3D printing is widely applied in various fields. For instance, in the process of making a metal laminated structure, the manufactured product has a porous structure. The medical science is also a field to which 3D printing is applied, especially in making medical artificial implants. However, because the metal laminates are mostly of a fixed structure, the metal laminates are not flexible.

TW patent No. 1536955 “BIONIC FIXING APPARATUS” discloses a flexible section with multiple holes. The flexible section can sustain a longitudinal force but does not have elasticity and is too rigid.

U.S. Pat. No. 9,918,849 “COILED IMPLANTS AND SYSTEMS AND METHODS OF USE THEREOF” discloses a connection of multiple C-shaped structures. However, the multiple C-shaped structures have weak shear force and cannot sustain the shear stress. Elasticity of the C-shaped structures cannot be adjusted according to the patient's physiological needs, and the C-shaped structures may have different diameters, resulting in decreased strength when adjusting the diameters.

### SUMMARY OF THE INVENTION

The main objective of the present invention is to provide a laminated structure with an adjustable elastic modulus which is applied to medical artificial implants.

The laminated structure with an adjustable elastic modulus has multiple connecting elements and at least one connecting point. Each connecting element is an S-shaped curved component, and has an inner end and an outer end. The multiple connecting elements are combined with each other by at least one connecting point, and a diameter of the at least one connecting point is equal to or greater than a diameter of each connecting element connected by the at least one connecting point.

Other objects, advantages, and novel features of the invention will become more apparent from the following detailed description when taken in conjunction with the accompanying drawings.

### BRIEF DESCRIPTION OF THE DRAWINGS

FIG. 1 is a perspective view of a first embodiment of a laminated structure with an adjustable elastic modulus in accordance with the present invention;

FIG. 2 is a front view of the laminated structure with an adjustable elastic modulus in FIG. 1;

FIG. 3 is a top view of the laminated structure with an adjustable elastic modulus in FIG. 1;

FIG. 4 is another perspective view of the laminated structure with an adjustable elastic modulus in FIG. 1;

FIG. 5 is another perspective view of the laminated structure with an adjustable elastic modulus in FIG. 1;

FIG. 6 is an operational perspective view of the laminated structure with an adjustable elastic modulus in FIG. 1;

FIG. 7 is an operational front view of the laminated structure with an adjustable elastic modulus in FIG. 1;

FIG. 8 is an operational top view of the laminated structure with an adjustable elastic modulus in FIG. 1;

FIG. 9 is an enlarged operational perspective view of the laminated structure with an adjustable elastic modulus in FIG. 1;

FIG. 10 is an operational perspective view of the laminated structure with an adjustable elastic modulus in FIG. 1;

FIG. 11 is a perspective view of a second embodiment of a laminated structure with an adjustable elastic modulus in accordance with the present invention;

FIG. 12 is another perspective view of the laminated structure with an adjustable elastic modulus in FIG. 11;

FIG. 13 is another perspective view of the laminated structure with an adjustable elastic modulus in FIG. 11;

FIG. 14 is another perspective view of the laminated structure with an adjustable elastic modulus in FIG. 11;

FIG. 15 is another perspective view of the laminated structure with an adjustable elastic modulus in FIG. 11;

FIG. 16 is another perspective view of the laminated structure with an adjustable elastic modulus in FIG. 11; and

FIG. 17 is another perspective view of the laminated structure with an adjustable elastic modulus in FIG. 11.

### DETAILED DESCRIPTION OF PREFERRED EMBODIMENT

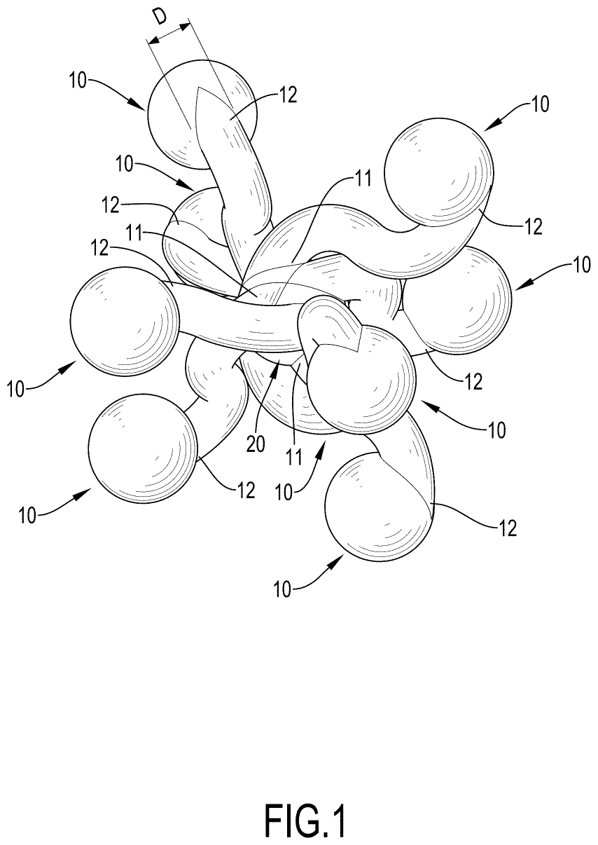

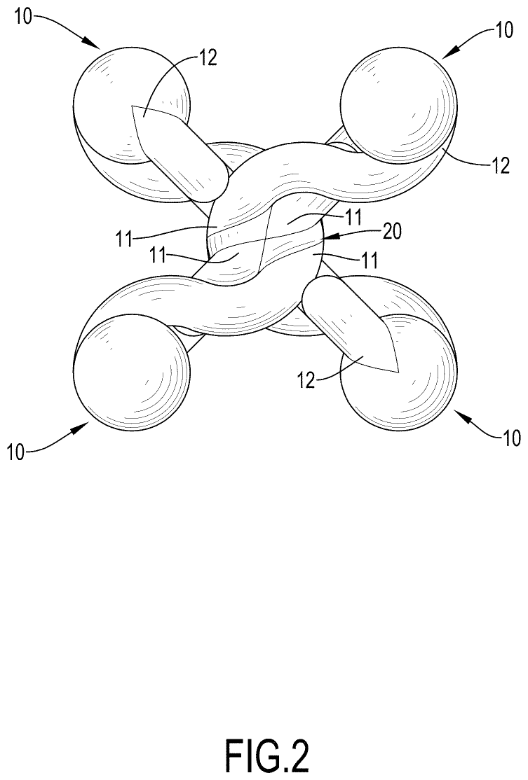

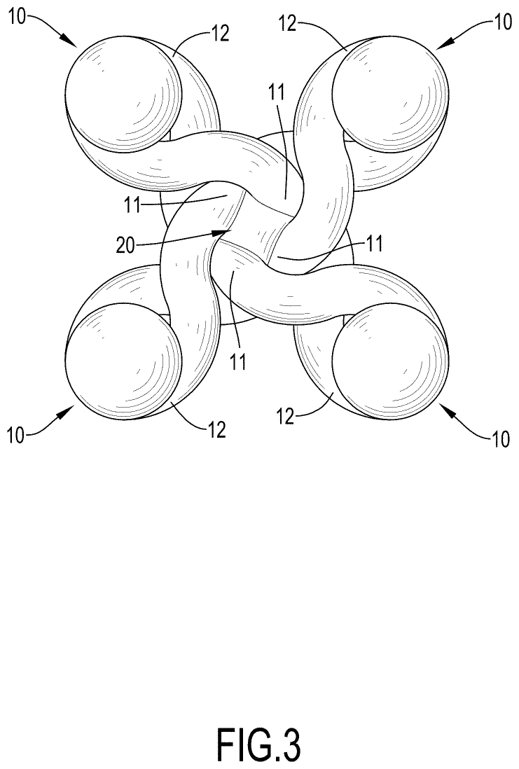

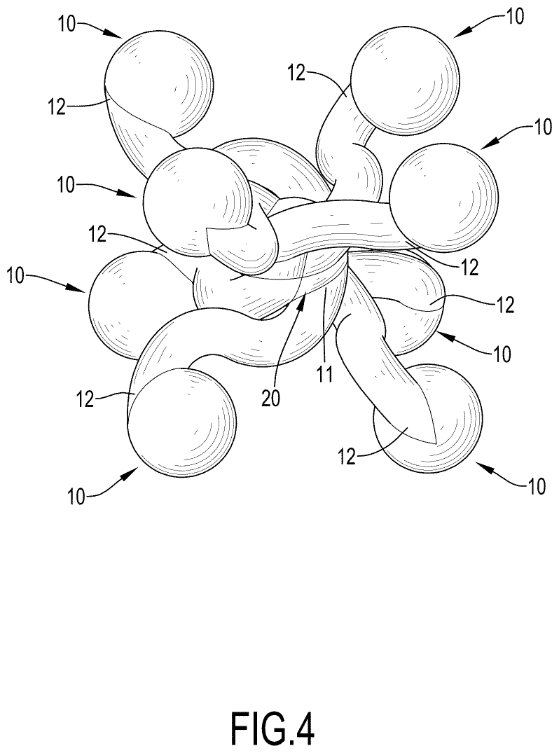

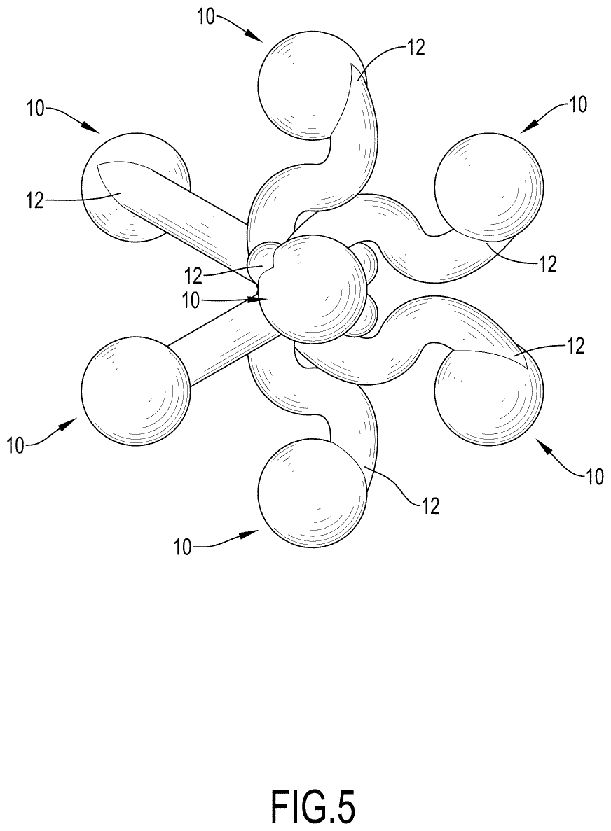

With reference to FIGS. 1 to 5, a first embodiment of a laminated structure with an adjustable elastic modulus in accordance with the present invention comprises multiple connecting elements 10 and at least one connecting point 20.

Each connecting element 10 is an S-shaped curved component, and has an inner end 11 and an outer end 12. Preferably, in the first embodiment, the laminated structure comprises eight connecting elements 10. The eight connecting elements 10 are combined with each other by a connecting point 20. A spherical structure is formed at the outer end 12 of each connecting element 10. Furthermore, the diameter of each connecting element 10 ranges from 0.2 mm to 3 mm. In the first embodiment, the diameter of each connecting element 10 ranges from 0.23 mm to 0.9 mm. Furthermore, the connecting point 20 may be a sphere, an ellipsoid, a cuboid, a cube, or may be composed of surfaces of different shapes.

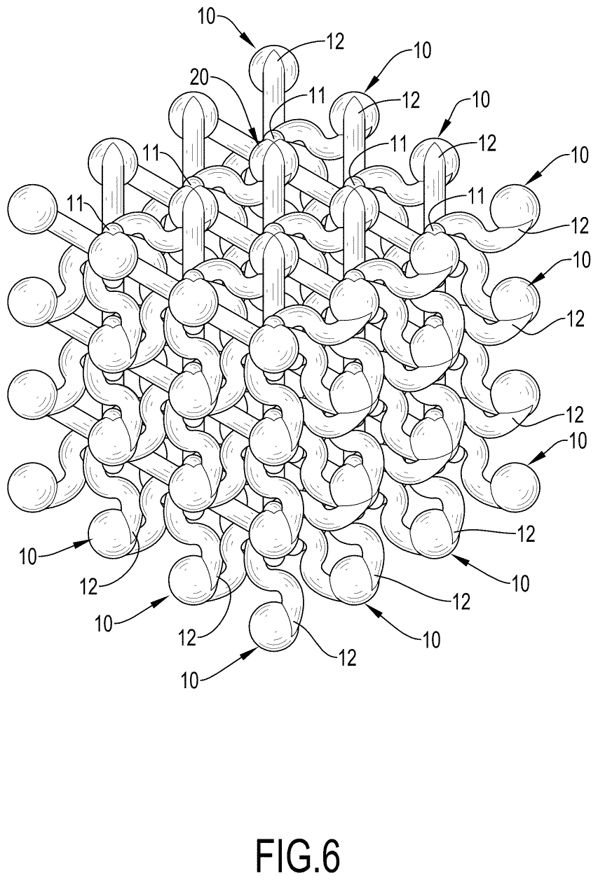

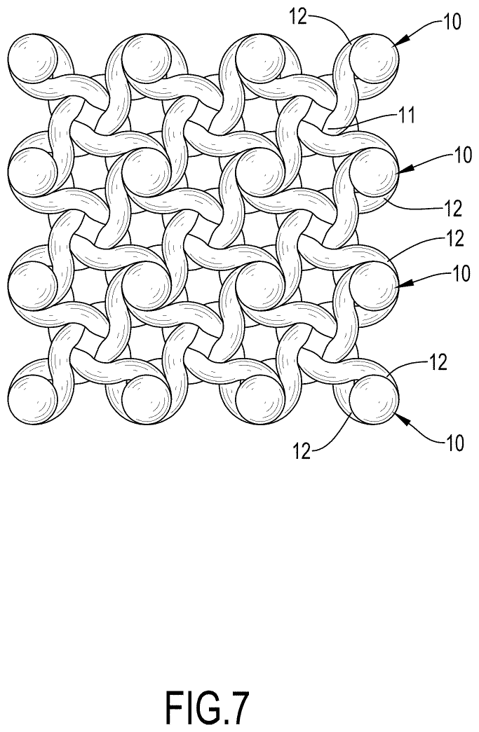

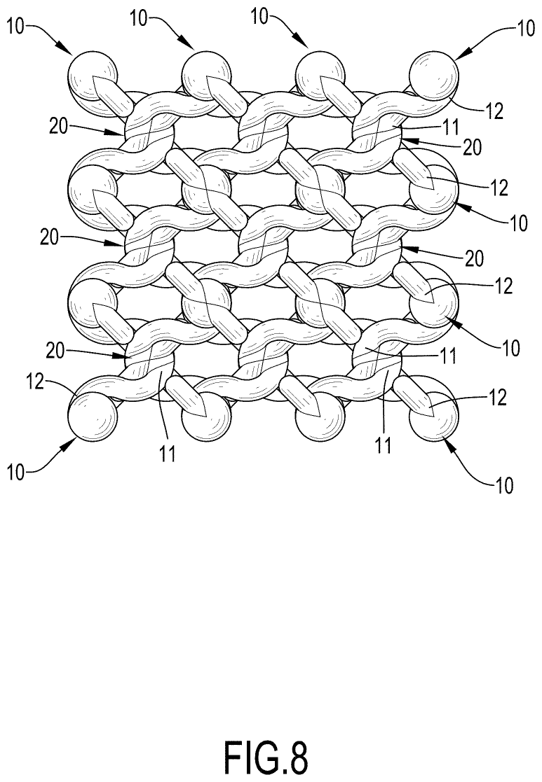

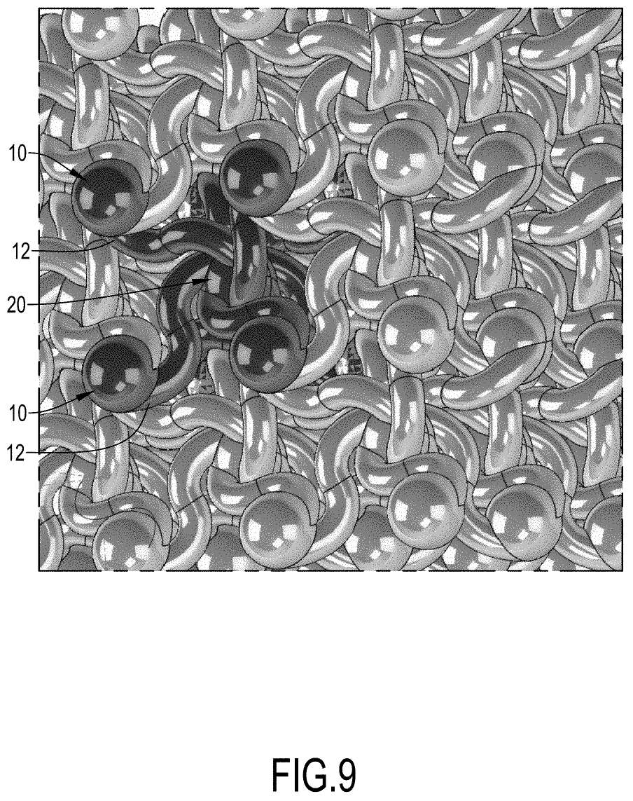

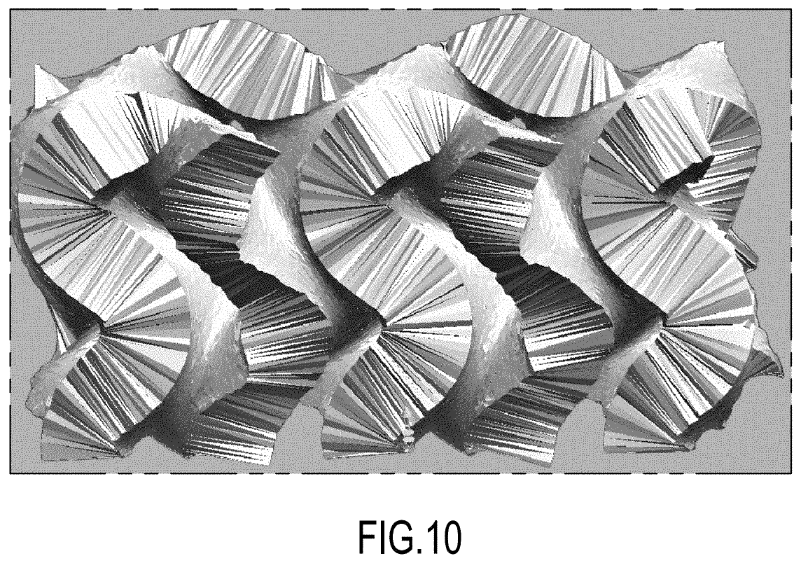

The first embodiment in accordance with the present invention is fabricated by powder-based 3D printing. With reference to FIGS. 6 to 10, when the first embodiment of the laminated structure with an adjustable elastic modulus in the present invention is in use, multiple connecting elements 10 are connected by at least one connecting point 20 to form a layered structure. The layered structure has a porous surface. The porous surface is specific to products made by powder-based 3D printing.

The structural strength of the laminated structure in accordance with the present invention is determined by the numbers of the connecting elements 10 and the at least one connecting point 20. Most of the time, one connecting point 20 is connected with 1 to 32 connecting elements 10. Preferably, in the first embodiment in accordance with the present invention, a connecting point 20 is connected with 1 to 16 connecting elements 10. The porosity and pore size of the laminated structure depend on the number of the connecting elements 10. Therefore, the elastic modulus of the first embodiment in accordance with the present invention is adjustable. The elastic modulus of the laminated structure in accordance with the present invention can be adjusted by adjusting the curvatures, the number of the curves at the same connection point, the diameter of the curve, the angle of the curve at the same point of intersection, etc.

With reference to FIGS. 6 to 10, when the laminated structure with an adjustable elastic modulus in accordance with the present invention is in use, the laminated structure is mounted in a human body. The muscle and blood of the human body would fill the pores of the laminated structure. In addition, the curvature of each connecting element 10 can be individually adjusted to form different elastic moduli. For instance, the connecting elements 10 at different positions have different curvatures to meet the individual needs of patients. Furthermore, the multiple connecting elements 10 can be combined with each other at different angles to meet the different requirements of tensile strength and compressive strength of different parts in different directions.

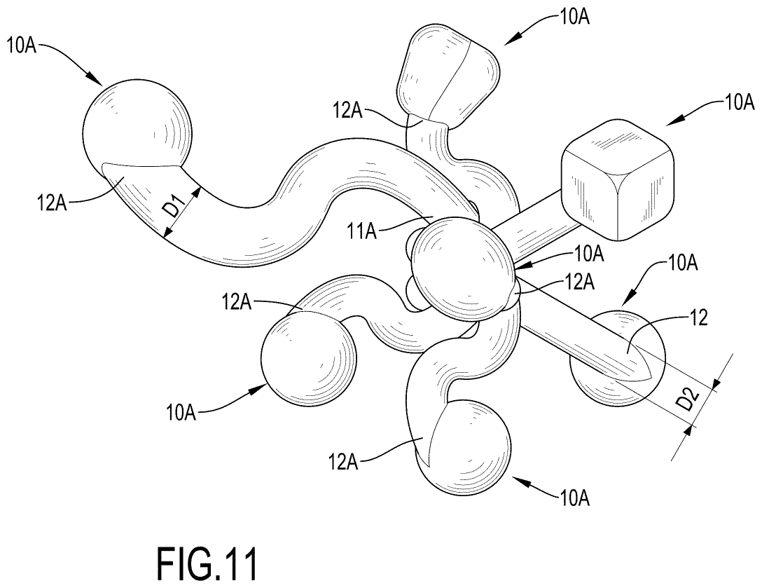

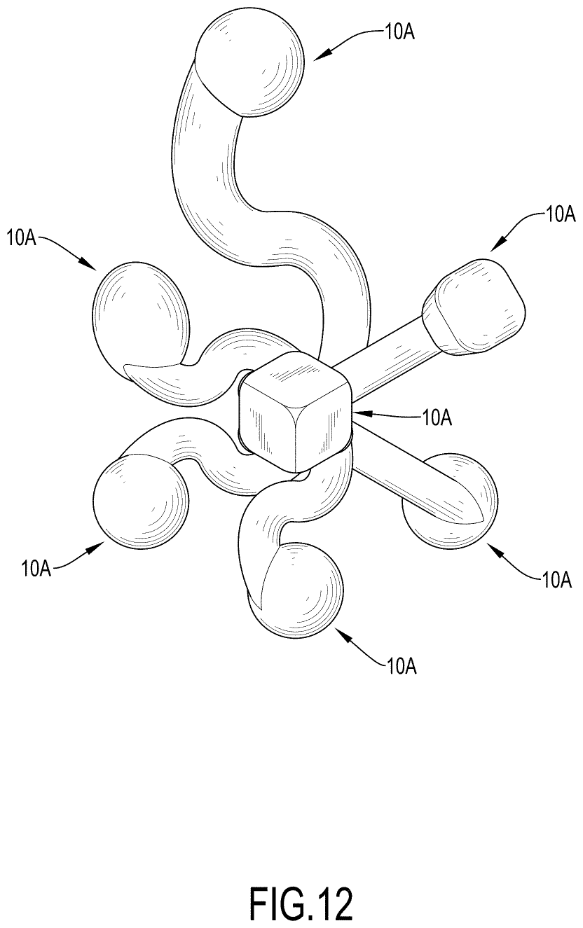

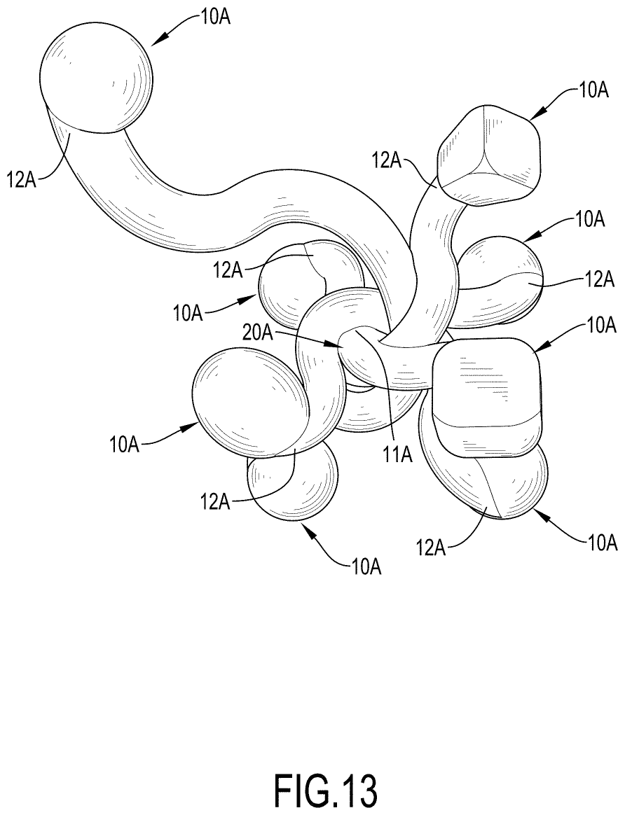

With reference to FIGS. 11 to 13, a second embodiment of a laminated structure with an adjustable elastic modulus in accordance with the present invention is substantially the same as the first embodiment except for the following features. The diameters of the connecting elements 10 are different from each other, and the curvatures of the connecting elements 10 are different from each other. Furthermore, a sphere, an ellipsoid, a cuboid, a cube, or multiple surfaces of different shapes may be formed at the outer end 12 of each connecting element 10.

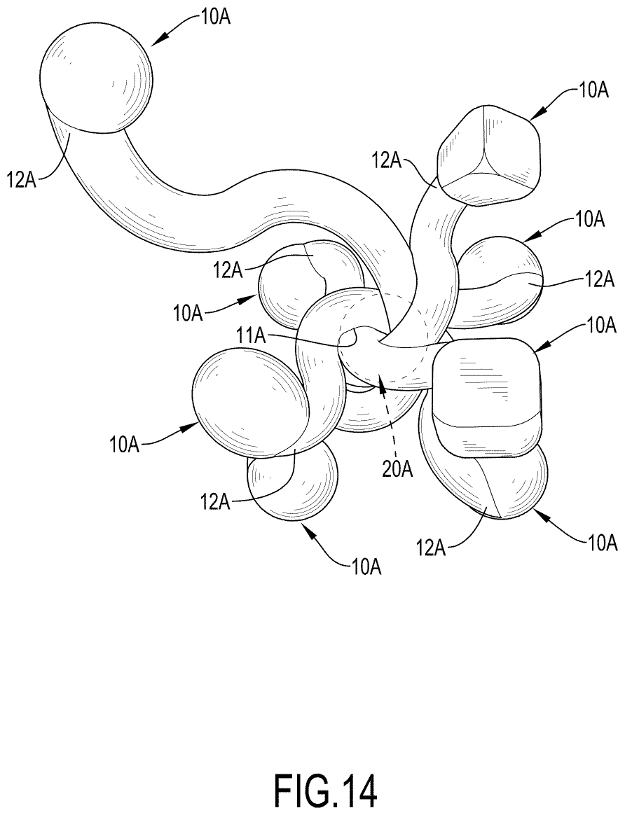

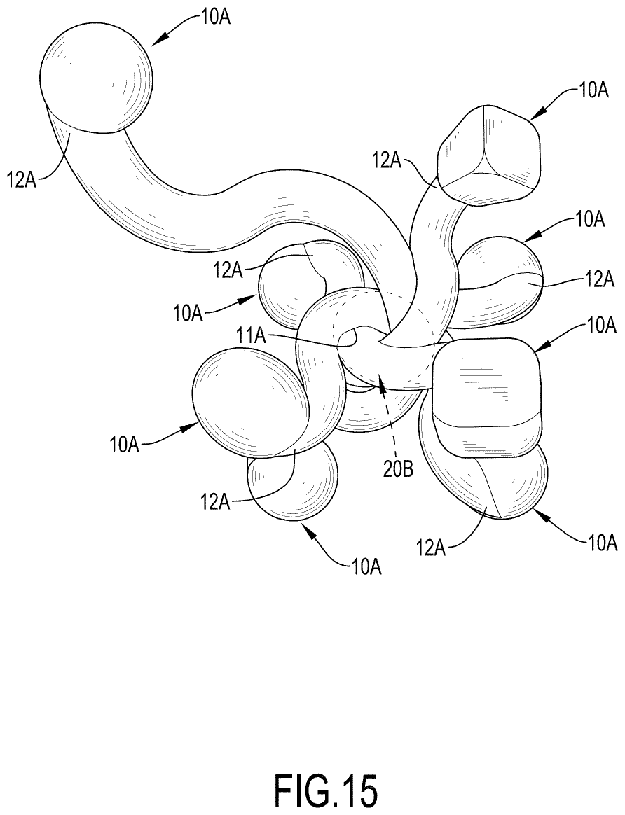

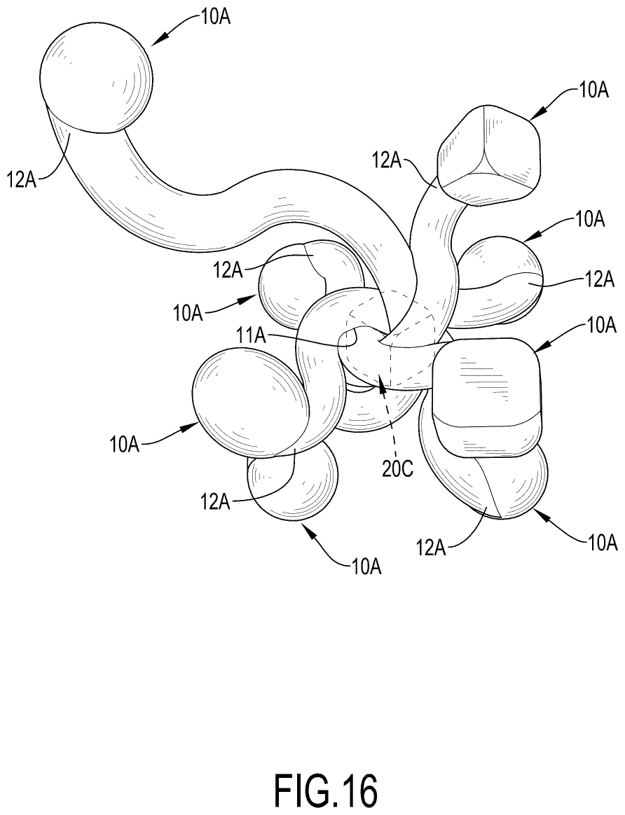

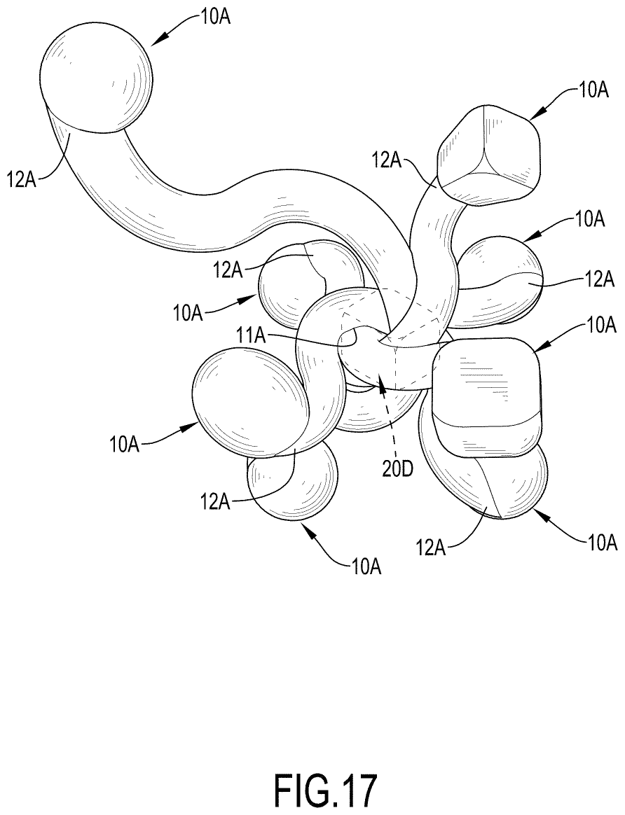

Furthermore, with reference to FIG. 14, the connecting point 20A is a sphere. With reference to FIG. 15, the connecting point 20B is an ellipsoid. With reference to FIG. 16, the connecting point 20C is a cube. With reference to FIG. 17, the connecting point 20D is a cuboid.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A laminated structure with an adjustable elastic modulus, the laminated structure comprising:
multiple connecting elements, each connecting element being an S-shaped curved component, and having an inner end and an outer end; and
at least one connecting point, the multiple connecting elements combined with each other by the at least one connecting point, and a diameter of the at least one connecting point being equal to or greater than a diameter of each connecting element connected by the at least one connecting point.

2. The laminated structure as claimed in claim 1, wherein the at least one connecting point is spherical, elliptical, cuboid, or cubic in shape.

3. The laminated structure as claimed in claim 2, wherein the multiple connecting elements are combined with each other by one said connecting point.

4. The laminate structure as claimed in claim 3, wherein a sphere, an ellipsoid, a cuboid, or a cube is formed at the outer end of each connecting element.

5. The laminated structure as claimed in claim 1, wherein the diameters of the connecting elements are different from each other.

6. The laminated structure as claimed in claim 2, wherein the diameters of the connecting elements are different from each other.

7. The laminated structure as claimed in claim 3, wherein the diameters of the connecting elements are different from each other.

8. The laminated structure as claimed in claim 4, wherein the diameters of the connecting elements are different from each other.

9. The laminated structure as claimed in claim 5, wherein curvatures of the connecting elements are different from each other.

10. The laminated structure as claimed in claim 6, wherein curvatures of the connecting elements are different from each other.

11. The laminated structure as claimed in claim 7, wherein curvatures of the connecting elements are different from each other.

12. The laminated structure as claimed in claim 8, wherein curvatures of the connecting elements are different from each other.
